(19) United States
(12) Patent Application Publication
(10) Pub. No.: US 2020/0254155 A1
(43) Pub. Date: Aug. 13, 2020

(54) FLUID REMOVAL SYSTEM

(71) Applicant: Bearpac Medical, LLC, Moultonborough, NH (US)

(72) Inventors: Theodore Karwoski, Moultonborough, NH (US); James Croteau, Brookline, NH (US); Jay Zimmerman, Somerville, MA (US)

(21) Appl. No.: 16/783,789

(22) Filed: Feb. 6, 2020

Related U.S. Application Data
(60) Provisional application No. 62/802,517, filed on Feb. 7, 2019.

(51) Int. Cl. A61M 1/00; A61M 25/09; A61M 25/00; A61M 39/10

(52) U.S. Cl. CPC A61M 1/0031; A61M 1/0029; A61M 1/0027; A61M 1/0019; A61M 25/09; A61M 2025/091; A61M 39/10; A61M 2205/3334; A61M 2205/502; A61M 2202/0014; A61M 2202/0492; A61M 25/0097

(57) ABSTRACT

A method and apparatus for fluid removal from a patient includes a disposable fluid removal subassembly and a portable drive subassembly that manage controlled extraction of a fluid from a patient. The fluid removal subassembly is configured for accessing a fluid filled cavity of a patient and also coupled with a fluid flow inducer having an inflow fluid intake and an outflow fluid output. The entire fluid removal subassembly, inclusive of a connector, a fluid conduit, and the fluid flow inducer, but exclusive of the outflow fluid output and a collection bag, is fluidly sealed from an external environment, and un-vented to the external environment. The fluid removal system enables a closed-loop fluid path between the patient through to the fluid flow inducer, which is under direct control by the patient of flow rate and therefore resulting pressure in the fluid path.

### CROSS-REFERENCE TO RELATED APPLICATION(S)

This application claims priority to, and the benefit of, co-pending U.S. Provisional Application No. 62/802,517, filed Feb. 7, 2019, for all subject matter contained therein. The disclosure of said provisional application is hereby incorporated by reference in its entirety.

### FIELD OF THE INVENTION

The present invention relates to a fluid removal system suitable for removing fluid from a patient. In particular, the present invention relates to a disposable fluid removal system configured for patient-controlled or caregiver-controlled extraction flow rates.

### BACKGROUND

Patients can develop excessive fluid on their lungs in the pleural space between the parietal pleura and visceral pleura. This pleural fluid accumulates over a short period of time, such as in days, and creates a sensation of drowning for the patient making it very difficult and uncomfortable to breath. Removal of this fluid is conventionally done via an indwelling catheter connected to a bottle that has been preset to a very high vacuum pressure that when activated removes or suctions out the fluid at a very low or high vacuum pressure and uncontrolled rate. There is virtually no control over this suction pressure and rate and as fluid is removed, the lung expands to fill the space and a significant amount of discomfort occurs for the patient. This accommodation of pressure differential as the lung expands and fluid is removed creates a clinical condition that is poorly understood and here to for never been effectively mitigated or controlled. The conventional devices that have been on the market for years either have no adjustments of either flow rate or pressure, or they do not provide an ability to adjust that rate based upon patient condition, amount of fluid being removed, or time of treatment, in real time.

### SUMMARY

There is a need for an improved disposable fluid removal system that can extract pleural or other fluids from a patient while under the real time control of the patient or a caregiver to specifically and accurately control flowrate and flow volume of the fluid extraction to manage, at a minimum, the pain experienced by the patient caused by the fluid removal. The present invention is directed toward further solutions to address this need, in addition to having other desirable characteristics.

Specifically, the present invention provides a small, precise, efficient fluid pump driven by a low voltage motor at various rates and settings to control the rate of fluid evacuation and corresponding intra cavity pressure induced into the pleural space of the patient for pain management purposes. There is a disposable fluid removal subassembly that couples ultimately with an indwelling catheter placed into the patient. The fluid collected is transferred from the fluid removal subassembly outlet through tubing to a graduated collection bag. The device of the present invention, via flow sensors, or electronically executed algorithms, gives an accurate estimate of the fluid volume that it has pumped as well as the graduations on the collection bag. The catheter connection, fluid conduit, fluid removal subassembly, and bag are disposable and for single use by the patient. The fluid removal subassembly connects to the top of the handheld portable drive subassembly, which contains advanced circuitry and controls for speed control, priming, pausing, stopping, and battery power.

In accordance with some embodiments, four different speeds from 25 cubic centimeters per minute (ccpm) to 50 ccpm to 75 ccpm to 100 ccpm are provided for fluid removal. These speeds allow for control of the negative pressure induced into the cavity as fluid is evacuated. As important as the technological advantages of the system is that, for the first time, the patient or the care provider has direct control of the therapy and flow volume and can instantly adjust the speed or pause the therapy/flow volume to control and minimize pain associated with the fluid removal.

In accordance with embodiments of the present invention, a fluid removal system includes a disposable fluid removal subassembly having a fluid conduit having a first end and a second end, a connector disposed at the first end of the fluid conduit, the connector configured for fluid coupling with a catheter coupling and catheter configured for extracting a target fluid inside a fluid filled cavity of a patient, and a fluid flow inducer disposed at the second end of the fluid conduit, the fluid flow inducer having an inflow fluid intake and an outflow fluid output. The entire fluid removal subassembly, inclusive of the connector, the fluid conduit, and the inflow fluid intake of the fluid flow inducer, but exclusive of the outflow fluid output, is fluidly sealed from an external environment, and un-vented to the external environment. A portable drive subassembly is provided and includes a drive configured for removable and replaceable engagement with the fluid flow inducer in such a way that activates the fluid flow inducer, and a control processor, wherein the portable drive subassembly is sized, dimensioned, and configured as a compact handheld device.

In accordance with aspects of the present invention, the system further includes a user interface in communication with the portable drive subassembly. The user interface comprises a display. A power level indicator can be included. A plurality of pressure controls can be configured to select a plurality of pressure settings. The connector can be sealingly coupled with the fluid conduit. The fluid removal subassembly can be configured for single-use.

In accordance with aspects of the present invention, the fluid removal system can be configured to maintain an adjustable controlled pressure. An internal pressure inside the disposable fluid removal subassembly can be controlled only by variation in rate of flow of fluid through the disposable fluid removal subassembly controlled only by rate of the fluid flow inducer. The fluid flow inducer can be an impeller. The fluid flow inducer can be one or more of gears, diaphragms, and/or pistons, motivating fluid flow.

In accordance with aspects of the present invention, an internal pressure of the fluid removal subassembly can be directly regulated without venting of air to or from outside of the fluid removal subassembly. Intra cavity pressure within the fluid filled cavity of the patient can be regulated only by variation of the drive of the portable drive subassembly.

In accordance with aspects of the present invention, the disposable fluid removal subassembly can generate unidirectional fluid flow from the first end of the conduit to the second end of the conduit and the fluid flow inducer, thereby preventing fluid back flow.

In accordance with aspects of the present invention, the portable drive subassembly can be disposable. The portable drive subassembly can be configured to be handheld. The portable drive subassembly can include a battery.

In accordance with aspects of the present invention, removal and/or replacement of the portable drive subassembly from and to engagement with the disposable fluid removal subassembly does not breach the fluidly sealed disposable fluid removal subassembly.

In accordance with aspects of the present invention, the system uses an electronically executed algorithm to monitor and record fluid volume, rate and prior therapies or treatments for the patient.

In accordance with embodiments of the present invention, a method of removing bodily fluid using a fluid removal system includes fluidly coupling a catheter to a fluid filled cavity of a patient, the catheter coupled with a disposable fluid removal subassembly having a fluid conduit having a first end and a second end, a connector disposed at the first end of the fluid conduit, and a fluid flow inducer disposed at the second end of the fluid conduit, the fluid flow inducer having an inflow fluid intake and an outflow fluid output. A portable fluid drive subassembly removably coupled with a fluid flow inducer is activated to pump fluid from the fluid filled cavity through the fluid flow inducer. Fluid from the fluid flow inducer is directed to a collection bag. An internal pressure caused by the fluid removal system at the fluid filled cavity of the user is directly proportional to a flowrate of the fluid through the fluid removal system which is directly managed and controlled by the patient in such a way that when the patient increases flowrate using a controller the suction pressure magnifies and when the patient decreases flowrate using the controller the suction pressure reduces.

### BRIEF DESCRIPTION OF THE FIGURES

These and other characteristics of the present invention will be more fully understood by reference to the following detailed description in conjunction with the attached drawings, in which:

FIG. 1 is a diagrammatic illustration of a fluid removal system;

FIG. 2 is an exploded view of a portable drive subassembly component of the fluid removal system;

FIG. 3 is an exploded view of a fluid flow inducer component, a catheter, and a fluid collection bag, of the fluid removal system; and

FIG. 4 is a diagrammatic illustration of the fluid removal system in use removing fluid from a patient.

### DETAILED DESCRIPTION

An illustrative embodiment of the present invention relates to a novel fluid removal system. The system includes a disposable fluid removal subassembly, having a fluid conduit and connector configured for accessing a fluid filled cavity of a patient and also coupled with a fluid flow inducer having an inflow fluid intake and an outflow fluid output. The entire fluid removal subassembly, inclusive of the connector, the fluid conduit, and the inflow fluid intake of the fluid flow inducer, but exclusive of the outflow fluid output, and an outflow catheter coupled to a collection bag, is fluidly sealed from an external environment, and un-vented to the external environment. A portable drive subassembly is configured for removable and replaceable engagement with the fluid flow inducer in such a way that activates the fluid flow inducer. A control processor manages operation of the portable drive subassembly to control flowrate and volume through the fluid flow inducer in response to user input from, e.g., a patient. The entire portable drive subassembly is sized, dimensioned, and configured as a compact handheld device. The structure of the fluid removal system enables a closed-loop fluid path environment between the fluid being removed from the patient through the fluid flow inducer, which is under direct control by the patient of flow rate and therefore resulting pressure in the fluid path, given there is no venting of air into that segment of the system. This differs from other known systems that include a relief valve or vent for releasing high negative pressure, with little to no control of the flowrate or pressure along the fluid path. A section between the fluid flow inducer and a fluid collection bag is low pressure and can be vented.

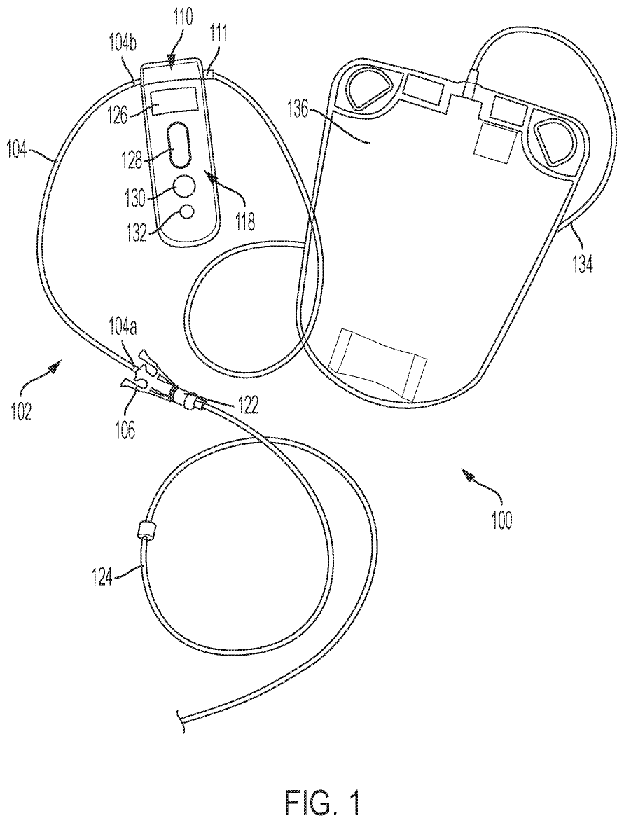

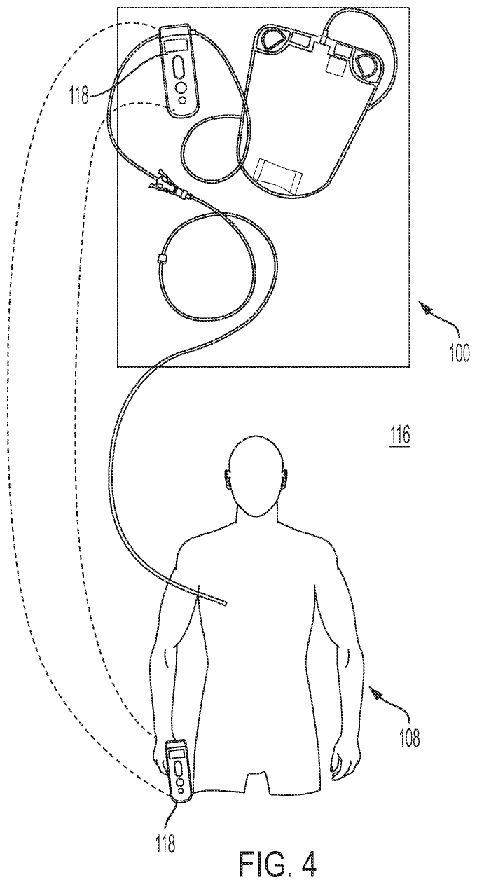

FIGS. 1 through 4, wherein like parts are designated by like reference numerals throughout, illustrate an example embodiment or embodiments of a fluid removal system, according to the present invention. Although the present invention will be described with reference to the example embodiment or embodiments illustrated in the figures, it should be understood that many alternative forms can embody the present invention. One of skill in the art will additionally appreciate different ways to alter the parameters of the embodiment(s) disclosed, such as the size, shape, or type of elements or materials, in a manner still in keeping with the spirit and scope of the present invention.

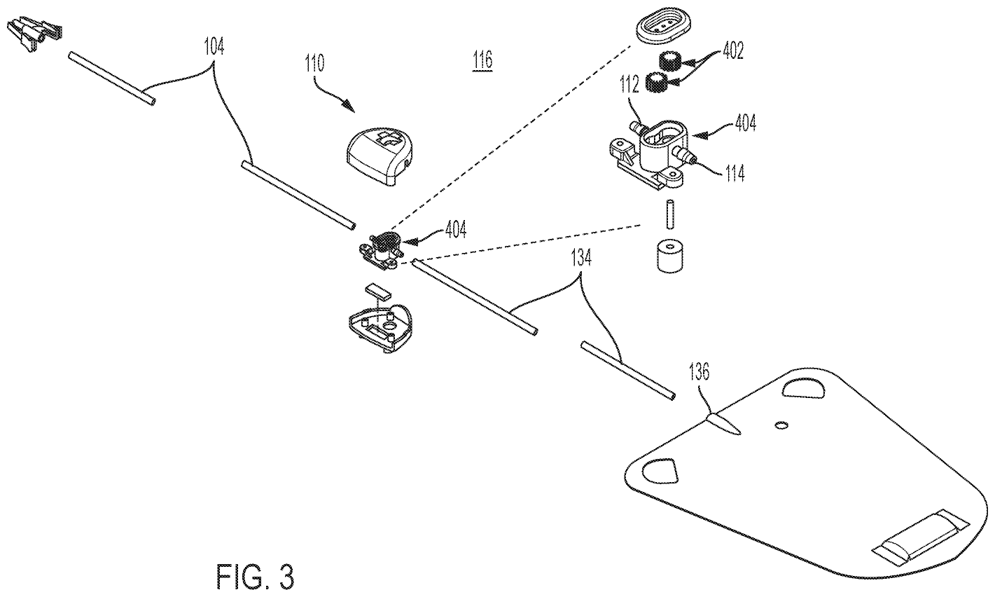

A fluid removal system 100 includes a disposable fluid removal subassembly 102. The disposable fluid removal subassembly 102 includes a fluid conduit 104 having a first end 104a and a second end 104b. A connector 106 is disposed at the first end 104a of the fluid conduit 104. The connector 106 is configured for fluid coupling with a catheter coupling 122 of a catheter 124 accessing a target fluid inside a fluid filled cavity of a patient 108. Further included in the disposable fluid removal subassembly 102 is a fluid flow inducer 110 (see also, FIG. 3) disposed at the second end 104b of the fluid conduit 104. The fluid flow inducer 110 has an inflow fluid intake 112 and an outflow fluid output 114. The entire fluid removal subassembly 102, inclusive of the connector 106, the fluid conduit 104, and the inflow fluid intake 112 of the fluid flow inducer 110, but exclusive of the outflow fluid output 114 and downstream conduit and collection bag 136, is fluidly sealed from an external environment, and un-vented to the external environment 116.

Also shown in the figure are an outflow fluid conduit 134. The outflow fluid conduit 134 fluidly couples the outflow fluid output 114 with a fluid collection bag 136 or any other fluid repository or disposal structure or process. Various equivalent structures may be utilized for the outflow fluid conduit 134 as well as the fluid collection bag 136, as would be appreciated by those of skill in the art.

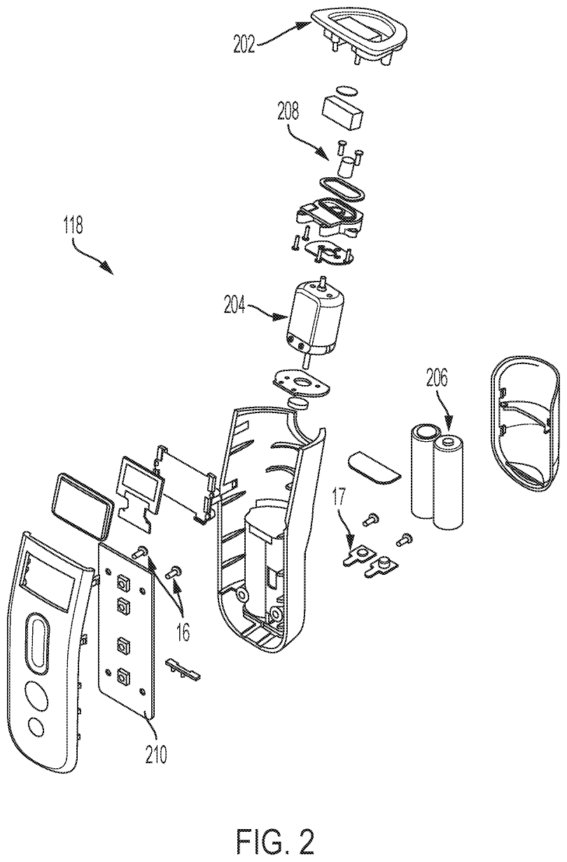

A portable drive subassembly 118 (see also, FIG. 2) includes a drive 120 configured for removable and replaceable engagement with the fluid flow inducer 110 in such a way that activates the fluid flow inducer 110. A control processor is in communication with the portable drive subassembly 118 and can access control and processor algorithms for implementing various desired functionality. This can include prescribing and controlling future therapy based upon past fluid volume extraction amounts, adjusting rate of therapy based upon analysis and consideration of prior therapies, and discomfort, prompting care givers and patients to anticipate end of therapy and fluid removal.

The portable drive subassembly 118 is sized, dimensioned, and configured as a compact handheld device, meaning it is easily held and operated by a single average adult-sized hand. The portable drive subassembly 118 is considered reusable for more than a single use, but also disposable so that the expectation is that once the particular patient 108 is done with their need for fluid removal treatments, the unit would be disposed of entirely in the normal course. As such, the portable drive subassembly 118 is a multi-use disposable device.

A user interface 126, such as an electronic display, is in communication with and/or a component of, the portable drive subassembly 118. The user interface 126 can provide a variety of information, including status of operation, power level, battery or charge level, and the like. A plurality of pressure controls can be configured to select a plurality of pressure settings, including a rate toggle 128 to increase or decrease flow rate, a pause button 130 to pause or restart operation of the device, and a power button 132 to power up or power down the portable drive subassembly 118. Accordingly, the present inventive fluid removal system 100 is structurally configured to provide direct control of pressure, flow rate, and overall flow volume, of fluid being removed from the patient, and that direct control enables superior pain management over prior conventional systems for fluid removal.

The connector 106 is sealingly coupled with the fluid conduit 104. A fluid conduit 104 as utilized herein is device as a pipe, tube, or the like, for conveying water or other fluid, in the present case the fluid is extracted fluid from a patient 108. The exploded view of the portable drive subassembly 118 in FIG. 2 shows all components of the device in accordance with an example embodiment, all of which will be evident to those of skill in the art based upon the illustrative figure, as such for purposes of conciseness, not all elements shall be called out herein. However, a few of the key components shown include an engagement component 202 for mechanically removably and replaceably engaging the portable drive subassembly 118 with the fluid removal subassembly 102. Also included are a motor 204, a drive 208, and a power supply 206 in the form of a battery with power supply contacts or spring plates 17, as well as a circuit board 210 including a processor with fasteners 16.

The fluid removal subassembly 102 is configured for single-use (for primarily sanitary reasons) and is disposable. The fluid removal system 102 is configured to maintain an adjustable controlled pressure. An internal pressure inside the disposable fluid removal subassembly 102 is controlled only by variation in rate of flow of fluid through the disposable fluid removal subassembly 102, which is controlled only by rate of the fluid flow inducer 110. The fluid removal subassembly is removably and replaceably mechanically coupled with the portable drive subassembly 118 at the engagement component 202.

The catheter coupling 122 of a catheter 124 accessing a target fluid inside a fluid filled cavity of a patient 108 is configured for quick connection or release in a fluid sealing manner with the connector 106. A valve seal plunger fits within a valve body. A valve spring fits over the plunger. A tubing connector is placed on top of the valve body and seals the aforementioned valve components in place within the valve body.

Turning again to FIG. 3, the fluid flow inducer 110 includes an impeller 402, which in the example illustrative embodiment is a dual impeller construct. The impeller 402 sits inside a body 404 of the fluid flow inducer 110. The fluid flow inducer 110 can be implemented in a number of different configurations, including one or more of gears, diaphragms, and/or pistons, motivating fluid flow.

An internal pressure of the disposable fluid removal subassembly 102 is regulated without venting of air to or from outside of the disposable fluid removal subassembly 102. The disposable fluid removal subassembly 102 is a closed-loop system. Intra cavity pressure within the fluid filled cavity of the patient 108 is directly regulated only by variation of the drive speed of the portable drive subassembly 118. With a faster drive speed, more fluid is pumped through the fluid flow inducer 110, and therefore a greater negative pressure or suction pressure is created on the fluid filled cavity of the patient, and a higher quantity per time of fluid is removed from the patient 108 in accordance with Bernoulli's Equation. Likewise, with a slower drive speed, less fluid is pumped through the fluid flow inducer 110, and therefore a lesser negative pressure or suction pressure is created on the fluid filled cavity of the patient, and a lower quantity per time of fluid is removed from the patient 108, again in accordance with Bernoulli's Equation. Example flow rates and pressures that can be implemented with the system and method of the present invention can range as follows, there can be, e.g., four different speeds from 25 cubic centimeters per minute (ccpm) to 50 ccpm to 75 ccpm to 100 ccpm are provided for fluid removal. Those of skill in the art will appreciate a number of different speed adjustments are possible, including for example, having ten different speed adjustments programed up to 250 ccpm.

The disposable fluid removal subassembly 102 generates unidirectional fluid flow from the first end 104a of the conduit to the second end 104b of the fluid conduit 104 and the fluid flow inducer 110, thereby preventing fluid back flow. A one-way valve can be incorporated therein as would be appreciated by those of skill in the art.

As is readily apparent by the figures, the portable drive subassembly 118 is configured to be handheld in that it is sized and dimensioned to fit and be comfortably grasped by an average adult human hand as would be appreciated by those of skill in the art. The portable drive subassembly 118 further comprises a power supply, such as e.g., a battery, rechargeable battery, an AC or DC input, or any other suitable power source or supply as would be appreciated by those of skill in the art.

Because of the configuration of the portable drive subassembly 118 and the disposable fluid removal subassembly 102, and the removable and replaceable coupling thereof, removal and/or replacement of the portable drive subassembly 118 from and to engagement with the disposable fluid removal subassembly 102 does not breach the fluidly sealed disposable fluid removal subassembly 102.

In accordance with aspects of the present invention, fluid removal system 100 uses one or more electronically executed algorithms carried out by a processor on the circuit board 210 to monitor and record fluid volume, rate and prior therapies or treatments for the patient 108. These algorithms can use electrical current parameters related to motor resistance, rotational speed, and duration, as well as power consumption to provide for controlling therapies and prescribing adjustments to future therapies.

In operation, the catheter 124 is inserted into the patient 108 to access a target fluid inside a fluid filled cavity of the patient 108. The catheter 124 is removably and sealingly coupled with the fluid conduit 104, which is in turn removably and sealingly coupled with the inflow fluid intake 112 of the fluid flow inducer 110. Likewise, the outflow fluid output 114 is coupled with the outflow fluid conduit 134 and the ultimate destination for the extracted fluid, such as the fluid collection bag 136.

Then with the portable drive subassembly 118 removably and replaceably coupled with the fluid flow inducer 110, the patient 108 and or user can activate the pumping operation by using the power button 132 and then the rate toggle 128 to increase or decrease the speed of the pumping operation by the fluid flow inducer 110. The fluid flow rate can be measured and quantified using one or more flow sensors 111, or other means as would be understood by those of skill in the art. As fluid is extracted from the patient 108, if there is no pain, the patient 108 is free to increase the flow rate via the rate toggle 128, thereby effecting faster removal of fluid in a given time. If the patient 108 is experiencing pain due to fluid being extracted too quickly, the rate toggle 128 can be used to decrease flow rate or even to pause entirely (using the pause button 130). With increased flowrate, there is increased suction (negative pressure) between the fluid filled cavity, the catheter 124, the fluid conduit 104, the inflow fluid intake 112 and the fluid flow inducer 110.

This entire subset of components of the system 100 are fluidly sealed with no venting or other release valves or the like, making control of the pressure within this subset entirely controlled by flowrate, which is under the direct control of the patient 108 via the portable drive subassembly 118 directly controlling the fluid flow inducer 110. Importantly, the patient 108 is able to extract a greater amount of fluid in lesser time if they are willing to increase flowrate to the maximum that is comfortable from a pain perspective, while simultaneously managing and controlling their pain experience due to the fluid removal, which differs from conventional systems that do not provide such capability. Likewise, should the patient 108 be experiencing pain, the present system 100 enables precise control at a lower flowrate, rather than requiring the patient to turn on flow or turn off flow in a more binary manner as with other conventional systems.

To any extent utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about” and “approximately” are intended to cover variations that may existing in the upper and lower limits of the ranges of subjective or objective values, such as variations in properties, parameters, sizes, and dimensions. In one non-limiting example, the terms “about” and “approximately” mean at, or plus 10 percent or less, or minus 10 percent or less. In one non-limiting example, the terms “about” and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. Within this specification, embodiments have been described in a way which enables a clear and concise specification to be written, but it is intended and will be appreciated that embodiments may be variously combined or separated without parting from the invention. It is intended that the present invention be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. A fluid removal system, comprising:
a disposable fluid removal subassembly, comprising:
a fluid conduit having a first end and a second end;
a connector disposed at the first end of the fluid conduit, the connector configured for fluid coupling with a catheter coupling of a catheter configured for extraction of target fluid inside a fluid filled cavity of a patient; and
a fluid flow inducer disposed at the second end of the fluid conduit, the fluid flow inducer having an inflow fluid intake and an outflow fluid output;
wherein an entirety of the fluid removal subassembly, inclusive of the connector, the fluid conduit, and the inflow fluid intake of the fluid flow inducer, but exclusive of the outflow fluid output, is fluidly sealed from an external environment, and un-vented to the external environment; and
a portable drive subassembly, comprising:
a drive configured for removable and replaceable engagement with the fluid flow inducer in such a way that activates the fluid flow inducer; and
a control processor;
wherein the portable drive subassembly is sized, dimensioned, and configured as a compact handheld device.

2. The fluid removal system of claim 1, further comprising a user interface in communication with the portable drive subassembly.

3. The fluid removal system of claim 2, wherein the user interface comprises a display.

4. The fluid removal system of claim 1, further comprising a power level indicator.

5. The fluid removal system of claim 1, further comprising a plurality of pressure controls configured to select a plurality of pressure settings.

6. The fluid removal system of claim 1, wherein the connector is sealingly coupled with the fluid conduit.

7. The fluid removal system of claim 1, wherein the fluid removal subassembly is configured for single-use.

8. The fluid removal system of claim 1, wherein the fluid removal system is configured to maintain an adjustable controlled pressure.

9. The fluid removal system of claim 1, wherein an internal pressure inside the disposable fluid removal subassembly is controlled only by variation in rate of flow of fluid through the disposable fluid removal subassembly controlled only by rate of the fluid flow inducer.

10. The fluid removal system of claim 1, wherein the fluid flow inducer comprises an impeller.

11. The fluid removal system of claim 1, wherein the fluid flow inducer comprises one or more of gears, diaphragms, and/or pistons, motivating fluid flow.

12. The fluid removal system of claim 1, wherein an internal pressure of the fluid removal subassembly is regulated without venting of air to or from outside of the fluid removal subassembly.

13. The fluid removal system of claim 1, wherein an intra cavity pressure within the fluid filled cavity of the patient is directly regulated only by variation of the drive of the portable drive subassembly.

14. The fluid removal system of claim 1, wherein the disposable fluid removal subassembly generates unidirectional fluid flow from the first end of the fluid conduit to the second end of the fluid conduit and the fluid flow inducer, thereby preventing fluid back flow.

15. The fluid removal system of claim 1, wherein the portable drive subassembly is disposable.

16. The fluid removal system of claim 1, wherein the portable drive subassembly is configured to be handheld.

17. The fluid removal system of claim 1, wherein the portable drive subassembly further comprises a battery.

18. The fluid removal system of claim 1, wherein removal and/or replacement of the portable drive subassembly from and to engagement with the disposable fluid removal subassembly does not breach the fluidly sealed disposable fluid removal subassembly.

19. The fluid removal system of claim 1, wherein the system uses an electronically executed algorithm to monitor and record fluid volume, rate and prior therapies or treatments for the patient.

20. The fluid removal system of claim 1, further comprising one or more flow sensors disposed to measure and quantify fluid flow through the system.

21. A method of removing bodily fluid from a patient using a fluid removal system, the method comprising:
fluidly coupling a catheter to a fluid filled cavity of a patient, the catheter coupled with a disposable fluid removal subassembly having a fluid conduit having a first end and a second end, a connector disposed at the first end of the fluid conduit, and a fluid flow inducer disposed at the second end of the fluid conduit, the fluid flow inducer having an inflow fluid intake and an outflow fluid output;
activating a portable fluid drive subassembly removably coupled with a fluid flow inducer to pump fluid from the fluid filled cavity through the fluid flow inducer; and
directing fluid from the fluid flow inducer to a collection bag;
wherein an internal pressure caused by the fluid removal system at the fluid filled cavity of the patient is directly proportional to a flowrate of the fluid through the fluid removal system which is directly managed and controlled by the patient in such a way that when the patient increases flowrate using a controller the suction pressure magnifies and when the patient decreases flowrate using the controller the suction pressure reduces.

22. The method of claim 21, wherein the patient directs control of the removal of fluid from the patient to actively manage pain and discomfort using the fluid removal system structurally configured to provide direct control of one or more of pressure, flow rate, and overall flow volume, measured and quantified using one or more flow sensors, by selecting one or more of a plurality of settings using a plurality of pressure controls

23. The method of claim 22, wherein the plurality of pressure controls comprise one or more of a rate toggle, a pause button, and a power button and selection is based on status of operation feedback from the one or more flow sensors provided through a user interface of the fluid removal system.

24. The method of claim 21, wherein one or more flow sensors monitor pressure inside the fluid removal subassembly of the fluid removal system and adjust the speed of the pumping operation by the fluid flow inducer and the portable fluid drive subassembly automatically to compensate for changes in pressure.
